(19) United States
(12) Patent Application Publication
(10) Pub. No.: US 2025/0161313 A1
(43) Pub. Date: May 22, 2025

(54) A METHOD FOR THE TREATMENT OF NEUROPATHY

(71) Applicants: INSTITUT NATIONAL DE LA SANTÉ ET DE LA RECHERCHE MÉDICALE, Paris (FR); UNIVERSITÉ DE LILLE, Lille (FR); CENTRE HOSPITALIER UNIVERSITAIRE DE LILLE, Lille (FR); BOARD OF TRUSTEES OF MICHIGAN STATE UNIVERSITY, East Lansing, MI (US)

(72) Inventors: David BLUM, Lille Cedex (FR); Christelle CAUFFIEZ, Lille Cedex (FR); Geoffroy LAUMET, East Lansing, MI (US)

(21) Appl. No.: 18/839,129

(22) PCT Filed: Mar. 7, 2023
(86) PCT No.: PCT/EP2023/055806

§ 371 Date: Aug. 16, 2024

(30) Foreign Application Priority Data
Mar. 8, 2022 (EP) 22305262.2

(51) Int. Cl. A61K 31/522; A61K 33/243; A61P 25/02

(52) U.S. Cl. CPC A61K 31/522; A61K 33/243; A61P 25/02

(57) ABSTRACT

Cisplatin is a potent chemotherapeutic drug, widely used in the treatment of various solid cancers. However, its clinical effectiveness is strongly limited by frequent severe adverse effects, such as neuropathy. Therefore, there is an urgent medical need to identify novel strategies limiting cisplatin-induced pain. Here, the inventors provide evidence that the FDA-approved adenosine A2A receptor antagonist istradefylline (KW-6002) significantly protects from cisplatin-induced neuropathy in experimental models of sub-chronic cisplatin treatment. In particular, the present invention relates to a method for the treatment of neuropathy (e.g. CIPN) in a subject in need therefore comprising administering to the subject a therapeutically effective amount of a selective A2A Adenosine Receptor (A2AR) antagonist.

### FIELD OF THE INVENTION

The invention relates to a method for the treatment of neuropathy in a subject in need therefore comprising administering to the subject a therapeutically effective amount of a selective A2A Adenosine Receptor (A2AR) antagonist.

### BACKGROUND OF THE INVENTION

Neuropathy is defined by the International Association for the Study of Pain (IASP) as a disturbance of function or pathological change in one or several nerves (mononeuropathy or polyneuropathy respectively). Neuropathy can be associated or not with pain (painless and painful neuropathy respectively). Neuropathy is induced by several factors such as, but not limited to, metabolic disorders (diabetic neuropathy), injury or disease affecting the nervous system (nerve trauma, stroke), genetic (Guillan-Barre syndrome, Charcot-Marie-Tooth disease), virus infection and antiretroviral drugs (HIV, HSV), and cancer chemotherapy. The latter is called Chemotherapy-Induced Peripheral Neuropathy (CIPN). CIPN can limit the dosage and selection of cytostatic drugs, delay further treatment cycles, or lead to discontinuation of cytostatic therapy. Therefore, altering the optimal chemotherapeutic treatment and reducing the chance of survival for cancer patients. The symptoms range from early post-treatment pain, such as the paclitaxel, to paraesthesias, sensory ataxia, numbness (loss of sensation), tingling, and mechanical and cold allodynia. The extremities of the body are predominantly affected, but neuropathy can proceed along the limbs during repetitive treatment. Substances that cause CIPN include the commonly used taxanes, platinum derivatives and vinca alkaloids, as well as the more rarely used ixabepilone, bortezomib and thalidomide.

There is no Food and Drug Administration (FDA)-approved treatment for CIPN. Identification of preventive or curative interventions and underlying mechanisms contributing to CIPN would be a significant step forward in improving cancer treatment adherence and the quality of life of cancer survivors.

Platinum-based compounds such as cisplatin, carboplatin, and oxaliplatin bind and damage DNA triggering cell death in rapidly dividing cells (e.g. cancer cells). These drugs are used in nearly all types of solid tumors. Though all three are known to cause classic symptoms of CIPN, higher incidences are seen with cisplatin and oxaliplatin. CIPN due to cisplatin is more often irreversible than in cases with oxaliplatin. CIPN is a dose-limiting toxicity with both cisplatin and oxaliplatin. While numerous potentially neuroprotective agents have been tested to reduce CIPN induced by cisplatin, none of these agents have proven the ability to prevent or limit the neurotoxicity of cisplatin.

Cisplatin is a potent antineoplastic agent widely used in the treatment of various solid cancers such as lung, ovarian or testicular cancers as well as certain forms of lymphomas (1). The anti-tumor action of cisplatin requires its intracellular bioactivation by the replacement of chlorides by water molecules, forming a highly reactive molecule that binds to DNA and induces cytotoxic lesions in tumors (2). However, the unwanted accumulation of cisplatin in healthy cells can also trigger genotoxicity. Indeed, the clinical use of cisplatin is restricted by various severe adverse effects, including chemotherapy-induced peripheral neuropathy (CIPN) (3-5).

In clinical practice, CIPN is often considered as a frequent but unavoidable adverse effect of cisplatin chemotherapy that should be accepted by patients (12). Therefore, there is an urgent medical need for strategies that alleviate peripheral neuropathy, without interfering with the efficiency of cisplatin to control tumor growth.

Adenosine plays a major role in cellular and tissue homeostasis (13-15). Its physiological function relies on four G-protein coupled receptors, A1, A2A, A2B, and A3 (16-19). Adenosine is important for several aspects of the physiology. In the present study, using mouse models of acute and sub-chronic cisplatin administration, the inventors serendipitously observed that the FDA-approved A2AR antagonist istradefylline (also known as KW-6002) mitigates pain hypersensitivity associated with CIPN, without altering but rather potentiating the anti-tumoral properties of cisplatin. These data support the repurposing of istradefylline as a valuable preventive approach in patients undergoing cisplatin treatment.

### SUMMARY OF THE INVENTION

The present invention relates to a method for the treatment of peripheral neuropathy induced by chemotherapy (CIPN) in a subject in need therefore comprising administering to the subject a therapeutically effective amount of a selective A2A Adenosine Receptor (A2AR) antagonist. In particular, the present invention is defined by the claims.

### DETAILED DESCRIPTION OF THE INVENTION

Cisplatin is a potent chemotherapeutic drug, widely used in the treatment of various solid cancers. However, its clinical effectiveness is strongly limited by frequent severe adverse effects, such as chemotherapy-induced peripheral neuropathy (CIPN). Therefore, there is an urgent medical need to identify novel strategies limiting neuropathy. Here, the inventors provide evidence that the FDA-approved adenosine A2A receptor antagonist istradefylline (KW-6002) significantly protects from neuropathic pain in an experimental model of sub-chronic cisplatin intoxication. Importantly, we also demonstrate that the anti-tumoral properties of cisplatin are not altered by istradefylline in tumor-bearing mice and could even be potentiated at the molecular level. Altogether, the present results support the use of istradefylline as a new valuable preventive approach for the clinical management of patients undergoing cisplatin treatment.

The present invention relates to a method for the treatment of neuropathy in a subject in need therefore comprising administering to the subject a therapeutically effective amount of a selective A_{2A} Adenosine Receptor (A_{2A}R) antagonist.

The present invention also relates to a method for the treatment of chemotherapy-induced peripheral neuropathy (CIPN) in a subject in need therefore comprising administering to the subject a therapeutically effective amount of a selective A_{2A} Adenosine Receptor (A_{2A}R) antagonist.

The present invention relates to a method for the treatment of neuropathy in a subject in need therefore comprising administering to the subject a therapeutically effective amount of istradefylline (KW-6002) or its derivatives.

The present invention also relates to a method for the treatment of chemotherapy-induced peripheral neuropathy (CIPN) in a subject in need therefore comprising administering to the subject a therapeutically effective amount of an istradefylline (KW-6002) or its derivatives.

The present invention also relates to a selective A2A Adenosine Receptor (A2AR) antagonist for use in the treatment of neuropathy in a subject in need thereof.

In some embodiment, the present invention relates the selective A2AR antagonist for use in the treatment of a chemotherapy-induced peripheral neuropathy in a subject in need thereof.

In some embodiment, the present invention relates to istradefylline (KW-6002) or its derivatives for use in the treatment of neuropathy in a subject in need thereof.

In some embodiment, the present invention relates to istradefylline (KW-6002) or its derivatives for use in the treatment of a chemotherapy-induced peripheral neuropathy in a subject in need thereof.

As used herein, the term “subject” refers to a mammal, such as a rodent, a feline, a canine, and a primate. Particularly, the subject according to the invention is a human. Particularly, the subject according to the invention is an adult. Particularly, the subject according to the invention is a child. Particularly, the subject according to the invention is a teenager. Particularly, the subject according to the invention is a newborn. As used herein, the term “subject” encompasses “patient”.

In some embodiment the subject of the present invention suffers from neuropathy.

As used herein the term “neuropathy”, also known as “peripheral neuropathy” refers to disease affecting the peripheral nerves, meaning nerves beyond the brain and spinal cord. Damage to peripheral nerves may induce pain, impair sensation, movement, gland or organ function depending on which nerves are affected; in other words, neuropathy affecting motor, sensory, or autonomic nerves result in different symptoms. More than one type of nerve may be affected simultaneously. Peripheral neuropathy may be acute (with sudden onset, rapid progress) or chronic (symptoms begin subtly and progress slowly), and may be reversible or permanent. Common causes include systemic diseases (such as diabetes or leprosy), hyperglycemia-induced glycation, vitamin deficiency, medication (e.g., chemotherapy, or commonly prescribed antibiotics including metronidazole and the fluoroquinolone class of antibiotics (such as ciprofloxacin, levofloxacin, moxifloxacin)), traumatic injury, including ischemia, radiation therapy, excessive alcohol consumption, immune system disease, celiac disease, non-celiac gluten sensitivity, or viral infection. It can also be genetic (present from birth) or idiopathic (no known cause).

In a particular embodiment, the neuropathy is a neuropathic pain or a painful neuropathy.

Neuropathic pain, also knows as painful neuropathy, is currently defined as pain initiated or caused by a primary lesion or dysfunction in the nervous system. Nerve damage can be caused by trauma and disease and thus the term “neuropathic pain” encompasses many disorders with diverse aetiologies. These include, but are not limited to, peripheral neuropathy, diabetic neuropathy, CIPN, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy and vitamin deficiency. Neuropathic pain is pathological as it has no protective role. It is often present well after the original cause has dissipated, commonly lasting for years, significantly decreasing a patient's quality of life (Woolf and Mannion, 1999, Lancet, 353, 1959-1964). The symptoms of neuropathic pain are difficult to treat, as they are often heterogeneous even between patients with the same disease (Woolf & Decosterd, 1999, Pain Supp., 6, S141-S147; Woolf and Mannion, 1999, Lancet, 353, 1959-1964). They include spontaneous pain, which can be continuous, and paroxysmal or abnormal evoked pain, such as hyperalgesia (increased sensitivity to a noxious stimulus) and allodynia (sensitivity to a normally innocuous stimulus).

Neuropathy affecting just one nerve is called “mononeuropathy” and neuropathy involving nerves in roughly the same areas on both sides of the body is called “symmetrical polyneuropathy” or simply “polyneuropathy”. When two or more (typically just a few, but sometimes many) separate nerves in disparate areas of the body are affected it is called “mononeuritis multiplex”, “multifocal mononeuropathy”, or “multiple mononeuropathy”.

Neuropathy may cause painful cramps, fasciculations (fine muscle twitching), muscle loss, bone degeneration, and changes in the skin, hair, and nails. Additionally, motor neuropathy may cause impaired balance and coordination or, most commonly, muscle weakness; sensory neuropathy may cause numbness to touch and vibration, reduced position sense causing poorer coordination and balance, reduced sensitivity to temperature change and pain, spontaneous tingling or burning pain, or skin allodynia (severe pain from normally nonpainful stimuli, such as light touch); and autonomic neuropathy may produce diverse symptoms, depending on the affected glands and organs, but common symptoms are poor bladder control, abnormal blood pressure or heart rate, and reduced ability to sweat normally.

Treatment may be for any purpose, including the therapeutic treatment of subjects suffering from neuropathy (e.g. chemotherapeutic-induced pain neuropathy), as well as the prophylactic treatment of subjects who do not suffer from pain (e.g., subjects identified as being at high risk pain).

In some embodiment the subject of the present invention suffers from chemotherapy-induced peripheral neuropathy.

In a particular embodiment, the method of the present invention is suitable for the treatment of chemotherapeutic-induced peripheral neuropathy (CIPN).

In a particular embodiment, the method of the present invention is suitable for the treatment of a painful neuropathy associated with a chemotherapeutic-induced peripheral neuropathy (CIPN).

As used herein the term “chemotherapy-induced peripheral neuropathy” (CIPN) refers peripheral neuropathy induced by antineoplastic drugs often called cancer chemotherapy. The International Association for the Study of Pain defines neuropathy as “a disturbance of function or pathological change in one of several nerves and may diffuse bilaterally. CIPN is a predominantly sensory neuropathy that may be accompanied by motor and autonomic changes. CIPN symptoms usually emerge late, that is, weeks or months after the completion of chemotherapy, with their severity being usually proportional to the cumulative dose of the drug. Some patients experience paradoxical worsening and/or intensification of symptoms after the cessation of treatment, as well as a phenomenon known as “coasting”, where either mild neuropathy worsens or new CIPN develops. Pain and sensory abnormalities may persist for months or even years after the cessation of chemotherapy.

Clinically, CIPN manifests itself as deficits in sensory, motor and/or autonomic functions of a varying intensity. Sensory symptoms usually develop first, involve the feet and hands and commonly present as a typical “glove and stocking” neuropathy with the most distal parts of the limbs exhibiting the greatest deficits. The symptoms comprise numbness, tingling, altered touch sensation, impaired vibration, paresthesias and dysesthesias induced by touch and warm or cool temperatures. Moreover, painful sensations, including spontaneous burning, shooting or electric shock-like pain as well as mechanical or thermal allodynia or hyperalgesia frequently occur. In severe cases, these symptoms can progress to a loss of sensory perception. Motor symptoms occur less frequently than sensory symptoms and, as a rule, assume the form of distal weakness, gait and balance disturbances, and impaired movements. These symptoms have a marked and often underappreciated impact on quality of life and safety, e.g., cancer patients who develop CIPN are three times more likely to fall. In severe cases, CIPN can lead to paresis, complete patient immobilization and severe disability. Sensory disorders occur more frequently than autonomic symptoms, which usually involve orthostatic hypotension, constipation and altered sexual or urinary function (77).

As used herein the term “cancer” has its general meaning in the art and refers to a group of diseases involving abnormal cell growth with the potential to invade or spread to other parts of the body. The term “cancer” further encompasses both primary and metastatic cancers. Examples of cancers that may treated by methods and compositions of the invention include, but are not limited to, cancer cells from the bladder, blood, bone, bone marrow, brain, breast, colon, esophagus, gastrointestinal, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; non encapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malign melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brennertumor, malignant; phyllodestumor, malignant; synovial sarcoma; mesothelioma, malignant; dysgerminoma; embryonal carcinoma; teratoma, malignant; strumaovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblasticodontosarcoma; ameloblastoma, malignant; ameloblasticfibrosarcoma; pinealoma, malignant; chordoma; glioma, malignant; ependymoma; ependymoma; protoplasmic astrocytoma; fibrillary astrocytoma; astroblastoma; medulloblastoma, glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocyticleukemia; mast cell leukemia; megakaryoblasticleukemia; myeloid sarcoma; and hairy cell leukemia.

As used herein, the terms “treatment” “treat,” and “treating” refer to reversing, alleviating, inhibiting the progress of a disease or disorder as described herein (i.e. pain), or delaying, eliminating or reducing the incidence or onset of a disorder or disease as described herein, as compared to that which would occur in the absence of the measure taken. The terms “prophylaxis” or “prophylactic use” and “prophylactic treatment” as used herein, refer to any medical or public health procedure whose purpose is to prevent the disease herein disclosed (i.e. pain). As used herein, the terms “prevent”, “prevention” and “preventing” refer to the reduction in the risk of acquiring or developing a given condition (i.e. pain), or the reduction or inhibition of the recurrence or said condition (i.e. pain) in a subject who is not ill, but who has been or may be near a subject with the condition (i.e. pain).

In some embodiments, the prophylactic methods of the invention are particularly suitable for subjects who are identified as at high risk for neuropathy and/or CIPN. Typically subject that are risk for pain include patient that will have a surgical operation.

Said compound of the present invention can be used as a drug. The compounds are useful for example in the treatment of neuropathy such as chemotherapeutic-induced peripheral neuropathy (CIPN).

As used herein, the term “A_{2A} Adenosine Receptor receptor” (A_{2A}R) also known as ADORA2A or RDC8 refers to an adenosine receptor, and also denotes the human gene encoding it. This protein is a member of the G protein-coupled receptor (GPCR) family which possess seven transmembrane alpha helices, as well as an extracellular N-terminus and an intracellular C-terminus. This protein plays an important role in many biological functions, such as heart rate and circulation, cerebral and renal blood flow, immune function, pain and sleep regulation. It has been implicated in pathophysiological states such as inflammatory diseases and disorders.

As used herein, the term “antagonist” or “inhibitor” refers to an agent (i.e. a molecule) which inhibits or blocks the activity of a receptor. For instance, an antagonist refers to a molecule which inhibits or blocks the activity of receptor.

As used herein, the term “biological activity” of A_{2A} Adenosine Receptor refers to a cisplatin-induced pain hypersensitivity associated with significantly downregulation of Tnf in spinal cord as well as Il6 and Tnf expression in the dorsal root ganglion (DRG).

Tests for determining the capacity of a compound to be selective A_{2A}R antagonist are well known to the person skilled in the art. In a preferred embodiment, the selective antagonist specifically binds to A_{2A}R in a sufficient manner to inhibit the biological activity of A_{2A}R. Binding to A_{2A}R and inhibition of the biological activity of A_{2A}R may be determined by any competing assays well known in the art. For example, the assay may consist in determining the ability of the agent to be tested as selective A_{2A}R antagonist to bind to A_{2A}R. The binding ability is reflected by the Kd measurement. The term “KD”, as used herein, is intended to refer to the dissociation constant, which is obtained from the ratio of Kd to Ka (i.e. Kd/Ka) and is expressed as a molar concentration (M). KD values for binding biomolecules can be determined using methods well established in the art. In specific embodiments, a selective antagonist that “specifically binds to A_{2A}R” is intended to refer to an inhibitor that binds to human A_{2A}R polypeptide with a K_{D} of 1 μM or less, 100 nM or less, 10 nM or less, or 3 nM or less. Then a competitive assay may be settled to determine the ability of the agent to inhibit biological activity of A_{2A}R. The functional assays may be envisaged such evaluating the ability cisplatin-induced pain hypersensitivity associated with significantly downregulation of Tnf in spinal cord as well as Il6 and Tnf expression in the dorsal root ganglion (DRG). The skilled in the art can easily determine whether a selective A_{2A}R antagonist neutralizes, blocks, inhibits, abrogates, reduces or interferes with a biological activity of A_{2A}R.

In some embodiment, the selective antagonist of the present invention (i.e. the selective A_{2A}R antagonists) the invention acts through direct interaction with the A_{2A} Adenosine Receptor.

As used herein, the term “neuropathy reducing agent” relate to a pharmaceutically acceptable compound or composition able to reduce, when administered to a subject, at least one parameter chosen among. In some the neuropathy reducing agent is a selective A2A Adenosine Receptor (A2AR) antagonist.

As used herein, the term “A_{2A} Adenosine Receptor (A_{2A}R) antagonist” refers to a selective antagonist of the A_{2A} Adenosine Receptor. In some embodiment, the selective A_{2A}R antagonists of the present invention can be used for reducing pain (e.g. neuropathy). These selective antagonists can also be used to reduce the neuropathy induced by one administration of a chemotherapeutical drug, as above-mentioned; the drug being active during several days after administration.

As used herein, the term “selective antagonist” is used as a shortcut for “selective A_{2A} Adenosine Receptors antagonist”. A selective antagonist according to the invention refers to an antagonist that specifically targets the receptors of A_{2A} Adenosine.

As used herein, the term “derivative” means that the derivative has been or may have been prepared from the underlying compound (i.e. selective A_{2A}R antagonists) and shares the core structural components.

As used herein, the term “analog” refers a compound that is structurally and functionally related to another compound; compounds and analogs share high structural similarity and have similar biological functions.

In some embodiment, the selective A_{2A} Adenosine Receptor antagonist is preferably chosen among istradefylline (KW-6002) or its derivatives, MSX-3, SCH-58261, NIR178 (also known as PBF-509), ciforadenant, AB928, AZD4635, EOS100850, Inupadenant (EOS-850), EXS21546, TT-10 and TT-53 and their pharmaceutically acceptable salts. Preferably, in all the embodiments, the selective A_{2A} Adenosine Receptor antagonist is preferably chosen among istradefylline (KW-6002) or its derivatives, MSX-3, SCH-58261, tozadenant (SYN 115) preladenant (SCH-420814), NIR178 (also known as PBF-509), ciforadenant, AB928, AZD4635, EOS100850, Inupadenant (EOS-850), EXS21546, TT-10 and TT-53. Exemplary of substituted purine compounds thereof act as selective adenosine A2a receptor (A2aR) antagonists or its derivatives include also the compounds disclosed in the International Patent Application WO2021179074. For example, WO2021179074 includes the following compounds:

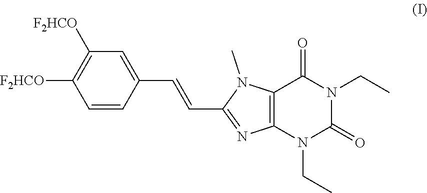 [SMILES: CCn1c(=O)c2c(nc(/C=C/c3ccc(OC(F)F)c(OC(F)F)c3)n2C)n(CC)c1=O]

or a pharmaceutically acceptable salt thereof

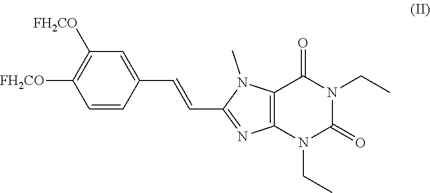

or a pharmaceutically acceptable salt

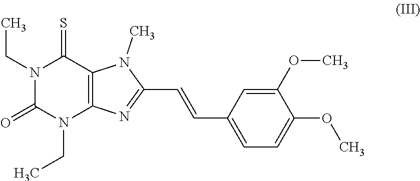 [SMILES: CCn1c(=S)c2c(nc(/C=C/c3ccc(OC)c(OC)c3)n2C)n(CC)c1=O]

or a pharmaceutically acceptable salt thereof.

These selective antagonists showed their efficiency in reducing neuropathy. They can be used alone, one after the other or in mixture of at least two antagonists.

In some embodiment, the present invention also relates to a selective A_{2A} Adenosine Receptor antagonist chosen among istradefylline (KW-6002) or its derivatives, MSX-3, SCH-58261, NIR178 (also known as PBF-509), ciforadenant, AB928, AZD4635, EOS100850, Inupadenant (EOS-850), EXS21546, TT-10 and TT-53 for use as a neuropathy reducing agent in the treatment of chemotherapeutic-induced pain neuropathy.

As used herein, the term “KW-6002” also known as Istradefylline means to 8-[(E)-2-(3,4-dimethoxyphenyl)vinyl]-1,3-diethyl-7-methyl-3,7-dihydro-1H-purine-2,6-dione. KW-6002 is having the following CAS number: 155270-99-8 and the following chemical formula:

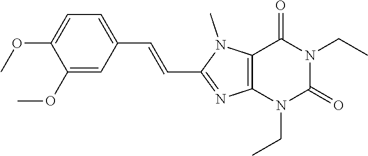 [SMILES: CCn1c(=O)c2c(nc(/C=C/c3ccc(OC)c(OC)c3)n2C)n(CC)c1=O]

As used herein, the term “MSX-3” refers to 3,7-Dihydro-8-[(1E)-2-(3-methoxyphenyl)ethenyl]-7-methyl-3-[3-(phosphonooxy)propyl-1-(2-propynyl)-1H-purine-2,6-dione disodium salt hydrate. MSX-3 is having the following CAS number 261717-23-1 and the following chemical formula:

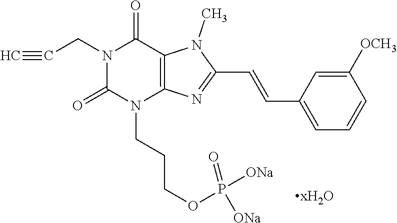

As used herein, the term “SCH-58261” refers to 2-(2-Furanyl)-7-(2-phenylethyl)-7H-pyrazolo[4,3-e][1,2,4]triazolo[1,5-c]pyrimidin-5-amine. SCH-58261 is having the following CAS number: 160098-96-4 and the following chemical formula:

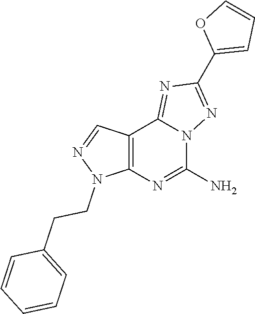 [SMILES: Nc1nc2c(cnn2CCc2ccccc2)c2nc(-c3ccco3)nn12]

As used herein, the term “Tozadenant” refers to 4-hydroxy-N-(4-methoxy-7-morpholin-4-yl-1,3-benzothiazol-2-yl)-4-methylpiperidine-1-carboxamide. Tozadenant is having the following CAS number: 870070-55-6 and the following chemical formula:

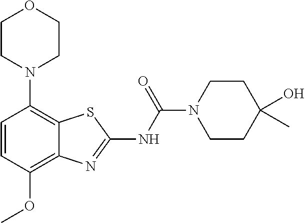 [SMILES: COc1ccc(N2CCOCC2)c2sc(NC(=O)N3CCC(C)(O)CC3)nc12]

As used herein, the term “Preladenant” refers to 2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine. Preladenant is having the following CAS number: 377727-87-2 and the following chemical formula:

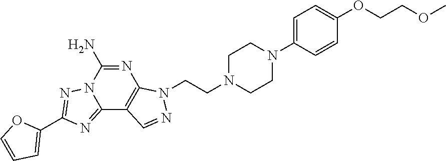 [SMILES: COCCOc1ccc(N2CCN(CCn3ncc4c3nc(N)n3nc(-c5ccco5)nc43)CC2)cc1]

As used herein, the term “NIR178” also known as “Taminadenant” or “PBF-509” refers to 5-bromo-2,6-di(pyrazol-1-yl)pyrimidin-4-amine. NIR178 is having the following CAS number: 1337962-47-6 and the following chemical formula:

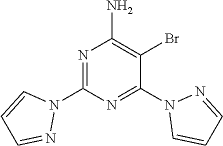 [SMILES: Nc1nc(-n2cccn2)nc(-n2cccn2)c1Br]

As used herein, the term “ciforadenant” refers to(S)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine. Ciforadenanis is having the following CAS number: 1202402-40-1 and the following chemical formula:

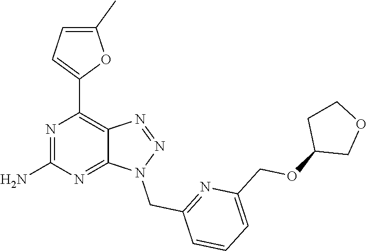 [SMILES: Cc1ccc(-c2nc(N)nc3c2nnn3Cc2cccc(CO[C@H]3CCOC3)n2)o1]

As used herein, the term “Etrumadenant” also known as “AB928” refers to 3-(2-amino-6-(1-((6-(2-hydroxypropan-2-yl)pyridin-2-yl)methyl)-1H-1,2,3-triazol-4-yl)pyrimidin-4-yl)-2-methylbenzonitrile. AB928 is having the following CAS number: 2239273-34-6 and the following chemical formula:

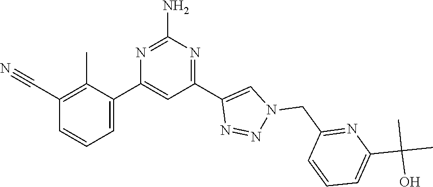 [SMILES: Cc1c(C#N)cccc1-c1cc(-c2cn(Cc3cccc(C(C)(C)O)n3)nn2)nc(N)n1]

As used herein, the term “Imaradenant” also knows as “AZD4635” refers to 6-(2-Chloro-6-methyl-4-pyridinyl)-5-(4-fluorophenyl)-1,2,4-triazin-3-amine. Imaradenant is having the following CAS number: 1321514-06-0 and the following chemical formula:

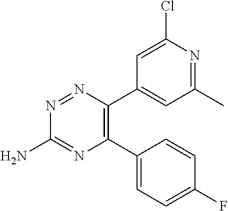 [SMILES: Cc1cc(-c2nnc(N)nc2-c2ccc(F)cc2)cc(Cl)n1]

As used herein, the term “EOS100850” refers to (+)-5-amino-3-{2-[4-(2,4-difluoro-5-{2-[(S)-methanesulfinyl]ethoxy}phenyl)piperazin-1-yl]ethyl}-8-(furan-2-yl)[1,3]thiazolo[5,4-e][1,2,4]triazolo[1,5-c]pyrimidin-2(3H)-one hydrochloride. EOS100850 is having the following CAS number: 2411004-22-1 and the following chemical formula:

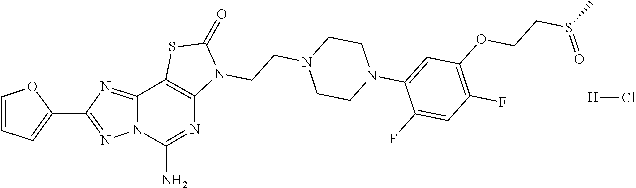

As used herein, the term “Inupadenant” also known as “EOS-850” refers to 7-amino-10-[2-[4-[2,4-difluoro-5-[2-[(S)-methylsulfinyl]ethoxy]phenyl]piperazin-1-yl]ethyl]-4-(furan-2-yl)-12-thia-3,5,6,8,10-pentazatricyclo[7.3.0.02,6]dodeca-1(9),2,4,7-tetraen-11-one. Inupadenant is having the following CAS number: 2246607-08-7 and the following chemical formula:

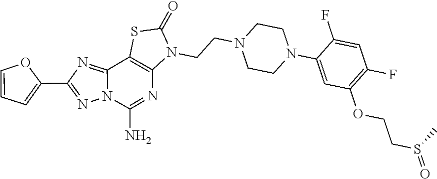 [SMILES: C[S@](=O)CCOc1cc(N2CCN(CCn3c(=O)sc4c3nc(N)n3nc(-c5ccco5)nc43)CC2)c(F)cc1F]

As used herein, the term “TT-10” refers to (2-(allylamino)-4-aminothiazol-5-yl)(5-fluorothiophen-2-yl)methanone. TT-10 is having the following CAS number: 2230640-94-3 and the following chemical formula:

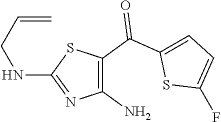 [SMILES: C=CCNc1nc(N)c(C(=O)c2ccc(F)s2)s1]

As used herein, the term “EXS21546” refers to a non-CNS penetrant A2AR-selective antagonist from Exscientia Limited (ClinicalTrials Identifier: NCT04727138).

As used herein, the term “TT-53” refers to selective Adenosine Receptor Antagonist from Tarus therapeutics.

The present invention also relates to the use of the selective A_{2A} Adenosine Receptor antagonist with combination with chemotherapeutic. In some embodiment, the therapeutic use of chemotherapeutic drug induces the occurrence of CIPN associated with pain hypersensitivity.

As used herein, the term “chemotherapy” has its general meaning in the art and refers to the treatment that consists in administering to the patient a chemotherapeutic agent. Chemotherapeutic agents include, but are not limited to alkylating agents such as thiotepa and cyclosphosphamide; alkyl sulfonates such as busulfan, improsulfan and piposulfan; aziridines benzodopa, carboquone, meturedopa, and uredopa; ethylenimines and such as methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-1 1); topoisomerase inhibitor RFS 2000; difluoromethylomithine (DMFO); retinoids such as retinoic acid; capecitabine; and pharmaceutically acceptable salts, acids or derivatives of any of the above.

As used herein, the term “chemotherapeutical drug” refers to any pharmaceutically acceptable composition being therapeutically or pharmaceutically efficient in cancer treatment. A chemotherapeutical drug can be a DNA-alkylating drug, for example.

In some embodiment, the chemotherapeutic drug is chosen among DNA-alkylating agents, particularly among platin-containing chemotherapeutic drugs and more particularly among chemotherapeutic drugs comprising an active compound chosen among cisplatin, carboplatin, oxaliplatin and the pharmaceutically acceptable salts thereof.

In particular, the therapeutic use of cisplatin is the occurrence of cisplatin-induced pain hypersensitivity (CIPN), in particular, pain hypersensitivity.

As used herein, the term “Cisplatin” refers to cis-diamminedichloroplatinum (II) and is a drug used in chemotherapy mainly because it blocks DNA synthesis and induces apoptosis through a mechanism of action via p53. Cisplatin is having the following CAS number: 15663-27-1 and the following chemical formula:

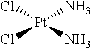

As used herein, the term “Oxaliplatin” refers to [(1R,2R)-cyclohexane-1,2-diamine] (ethanedioato-O,O′) platinum(II). Oxaliplatin is having the following CAS number: 63121-00-6 and the following chemical formula:

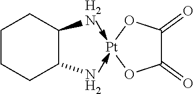

As used herein, the term “Carboplatin” refers to cis-diamine (cyclobutane-1,1-dicarboxylate-O,O′) platinum(II). Carboplatin is having the following CAS number: 41575-94-4 and the following chemical formula:

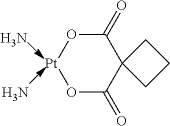

As used herein, the terms “able to induce overexpression of A_{2A} Adenosine Receptors” means that when the drug is administrated in a given amount, in a pharmaceutically effective amount or dosage, for example, the relative expression of A_{2A} Adenosine Receptors is over 1-fold and more particularly over 1.1-fold.

As used herein the terms “administering” or “administration” refer to the act of injecting or otherwise physically delivering a substance as it exists outside the body (e.g. selective A_{2A} Adenosine Receptor (A_{2A}R) antagonist) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

A “therapeutically effective amount” refers to an amount effective, at dosages and for periods of time necessary, to achieve a desired therapeutic result. A therapeutically effective amount of drug may vary according to factors such as the disease state, age, sex, and weight of the individual, and the ability of drug to elicit a desired response in the individual. A therapeutically effective amount is also one in which any toxic or detrimental effects of the antibody or antibody portion are outweighed by the therapeutically beneficial effects. The efficient dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. One of ordinary skill in the art would be able to determine such amounts based on such factors as the subject's size, the severity of the subject's symptoms, and the particular composition or route of administration selected. An exemplary, non-limiting range for a therapeutically effective amount of drug is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. As non-limiting examples, treatment according to the present invention may be provided as a daily dosage of the agents of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, per days, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

In some embodiment the therapeutically effective amount of the selective A_{2A} Adenosine Receptor antagonist, can be equal to 10 mg or more and equal to 350 mg or less and particularly equal to 20 mg, 21 mg, 30 mg, 35 mg, 40 mg, 60 mg, 70 mg, 100 mg or 120 mg, 160 mg, 180 mg, 210 mg or 240 mg. In a particular embodiment, when the selective A_{2A} Adenosine Receptor antagonist is KW-6002 or its derivatives, its therapeutically effective amount is preferably under a maximum value, this value can be equal to or more than 60 mg to 230 mg and more preferably equal to 70 mg or more and equal to 210 mg or less. As herein after explained, when the amount of this particular selective antagonist is over a threshold, it has no more activity as a pain-reducing agent (e.g. neuropathy reducing agent), and particularly in reducing pain induced by cisplatin (e.g. neuropathy induced by cisplatin).

In some embodiment, the therapeutically effective amount of the chemotherapeutical drug can be equal to 80 mg or more and equal to 1400 mg or less and particularly can be equal to 140 mg, 147 mg, 175 mg, 630 mg, 692 mg and 700 mg. Values particularly suitable when the chemotherapeutic drug comprises as an active compound cisplatin and particularly when said drug comprises as the sole active compound cisplatin are 140 mg, 700 mg and 1400 mg.

As used herein, the term “combination” is intended to refer to all forms of administration that provide a first drug together with a further (second, third . . . ) drug. The drugs may be administered simultaneously, separately or sequentially and in any order. According to the invention, the drug is administered to the subject using any suitable method that enables the drug to reach the chondrocytes of the bone growth plate. In some embodiments, the drug administered to the subject systemically (i.e. via systemic administration). Thus, in some embodiments, the drug is administered to the subject such that it enters the circulatory system and is distributed throughout the body. In some embodiments, the drug is administered to the subject by local administration, for example by local administration to the growing bone.

As used herein, the terms “combined treatment”, “combined therapy” or “therapy combination” refer to a treatment that uses more than one medication. The combined therapy may be dual therapy or bi-therapy.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

In some embodiment, the selective A_{2A} Adenosine Receptor antagonist is to be administrated before administration of said chemotherapeutic drug. and preferably, at least one hour before said chemotherapeutical drug administration.

In some embodiment, the chemotherapeutic drug can be administrated in one administration. In this case, the selective antagonist can be administrated before the chemotherapeutic drug administration, preferably at least one hour before. It can also be administrated before and after the chemotherapeutic drug administration. For example, it can be administrated at least one time, the day after the chemotherapeutic drug administration and for example, once a day over one day, two days, five days or 8 days after the chemotherapeutic drug administration. When the chemotherapeutic drug and particularly when the chemotherapeutic drug comprises as an active compound cisplatin and preferably cisplatin as the sole anticancer active compound and as the sole active compound, is administrated several times, once a day over several days, for example (one a day, over a week, for example), the selective antagonist is preferably administrated before each administration of the chemotherapeutic drug, and for example, at least one hour before. The selective antagonist can also be administrated once a day after the last administration of said chemotherapeutic drug and particularly over at least one day, or two days after the last administration of said chemotherapeutic drug and for example, over 5 days after the last administration of said chemotherapeutic drug.

The present invention relates to a therapeutically effective amount of a combination of a selective A_{2A} Adenosine Receptor (A_{2A}R) antagonist and chemotherapeutic drug for use in the treatment of neuropathy (e.g. CIPN).

In a particular embodiment, the invention relates to a therapeutically effective amount of a combination of KW-6002 or its derivatives and cisplatin for use in the treatment of neuropathy (e.g. CIPN).

The present invention also relates to a i) a selective A_{2A} Adenosine Receptor (A_{2A}R) antagonist and ii) chemotherapeutic drug for simultaneous, separate or sequential use in the treatment of neuropathy (e.g. CIPN).

In a particular embodiment, the invention relates to a i) KW-6002 or its derivatives and ii) cisplatin for simultaneous, separate or sequential use in the treatment of neuropathy (e.g. CIPN).

The present invention also relates to a pharmaceutical composition comprising a therapeutically effective amount of a selective A_{2A} Adenosine Receptor (A_{2A}R) antagonist and a chemotherapeutic drug. The pharmaceutical composition according to the invention wherein the A_{2A} Adenosine Receptor (A_{2A}R) antagonist is KW-6002 or its derivatives and wherein the chemotherapeutic drug is cisplatin.

More particularly, the pharmaceutical composition according to the invention is suitable for treating neuropathy (e.g. chemotherapeutic-induced pain neuropathy).

The selective A_{2A} Adenosine Receptor (A_{2A}R) antagonist and/or the chemotherapeutic drug as defined above and the pharmaceutical combination according to the invention, as described above may be combined with pharmaceutically acceptable excipients, and optionally sustained-release matrices, such as biodegradable polymers, to form pharmaceutical compositions.

The selective A_{2A} Adenosine Receptor (A_{2A}R) antagonist and/or the chemotherapeutic drug as described above may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, parenteral, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal, parenteral and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The invention will be further illustrated by the following figures and examples. However, these examples and figures should not be interpreted in any way as limiting the scope of the present invention.

### FIGURES

FIG. 1. Preventive treatment of KW-6002 reduces pain hypersensitivity and cytokine upregulation induced by cisplatin. (A) Mechanical sensitivity measured by von Frey hairs in mice in response to cisplatin and/or KW-6002 in the sub-chronic paradigm described on FIG. 2. The arrow represents cisplatin and/or PBS injection. Data are shown as mean±SEM. ^{∘∘∘}p<0.001 vs. Cisplatin (n=5/group; Two-Way ANOVA followed by Tukey's post-hoc test). mRNA levels of Il6 and Tnf in spinal cord (B-C) and DRG (D-E) 8 days after the start of cisplatin treatment. Data are shown as mean±SEM. ^{∘}p<0.05 vs. Cisplatin (n=5/group; One-Way ANOVA followed by Tukey's post-hoc test). (F and G) mRNA levels of Il 1b (F) and Ccl2 (G) in DRGs 8 days after the start of cisplatin treatment. Data are the mean±SEM. *P<0.05 versus vehicle; ^{∘∘}P<0.01 versus cisplatin; 2-way ANOVA (n=5/group).

FIG. 2. Schematic representation of the animal procedure. KW-6002 administration schedule in the sub-chronic model. C57BL6/J 8-weeks old male mice were randomized to 4 groups: Vehicle, KW-6002, Cisplatin or KW-6002/Cisplatin. Cisplatin administration was (3 mg/kg) for 6 days and were sacrificed 72 h after the last injection. The first administration of KW-6002 (3 mg/kg) was performed five days prior cisplatin treatment and daily until the sacrifice.

FIG. 3. KW6002 prevents nephrotoxicity and neurotoxicity without attenuating the antitumoral properties of cisplatin in the mEERL syngeneic in vivo mouse model. (A-C) KW6002 alleviated cisplatin-induced nephrotoxicity as estimated by mRNA levels of KIM1 (A) and the inflammatory markers Tnfa and Il6 (B and C). *P<0.05 and ***P<0.001 versus vehicle; ^{∘}P<0.01 and ^{∘∘}P<0.01 versus cisplatin; 1-way ANOVA (n=5-6/group). (D) Mechanical sensitivity measured by von Frey hairs in mice in response to cisplatin and/or KW6002. The arrow represents cisplatin and/or PBS injection. Data are the mean±SEM. ***P<0.001 versus vehicle; ^{∘∘∘}P<0.001 versus cisplatin; 2-way ANOVA (n=5/group). (E) Absolute tumor sizes in animals in the different groups. Results indicate the mean±SEM. ***P<0.001 versus vehicle; ^{∘}P<0.05 versus cisplatin; 2-way ANOVA (n=5-6 animals/group).

### EXAMPLE

### Material & Methods

### Animals and Treatments.

Animal experiments were adapted from 63-64. Animal procedures were performed in 8 to 10 weeks old male C57B16/J mice (Janvier Labs except for pain experiments, Jackson lab). Mice were fed a laboratory standard diet with water and food ad libitum and were kept under constant environmental conditions with a 12-hour light-dark cycle. Istradefylline (KW-6002; Tocris) was dissolved in a carrier solution consisting in 15% DMSO, 15% cremophor (Sigma), 70% saline solution (vehicle). Cisplatin (Accord Healthcare) was dissolved in saline solution. Neuropathy resulting from of sub-chronic cisplatin was evaluated following six daily i.p. injections of cisplatin (3 mg/kg) starting at day 0 and mice were sacrificed 72h after the last injection of cisplatin (day 8). KW-6002 was administered daily i.p. from day −5 to day 7 (FIG. 2).

mEERL In Vivo Tumor Model.

We used a validated murine model of HPV+ oropharyngeal squamous cell carcinoma as previously described (noncommercial) (80). This model consists of oropharyngeal epithelial cells from C57Bl/6 male mice that stably express the HPV16 viral oncogenes E6 and E7, H-Ras, and luciferase (mEERL cells). mEERL cells were grown in a T75 flask until confluent, after which cells were trypsinized and harvested, washed 3 times with sterile PBS, and resuspended in 1 mL sterile PBS to the appropriate concentration. Mice were injected s.c. into the right flank with 20 μL solution containing either 1,000,000 mEERL cells or PBS (vehicle). The day of mEERL cell injection is indicated as day −14. Tumor volume was monitored using Vernier digital calipers. When the tumor volume reached 100 mm3, the mice were randomly ascribed to 1 of the 3 experimental groups (vehicle; cisplatin; or cisplatin plus KW6002).

### Behavioral Assessment.

Mechanical pain sensitivity, a classical read out for CIPN in rodents, was assessed using von Frey filaments as previously described (65-66). Briefly, mice were placed in opaque boxes (10×10×10 cm). After a 30 min habituation period, von Frey filaments were applied, and the paw withdrawal threshold was calculated using the “up & down” method. Behavioral testing was performed by experimenter blinded to treatment and genotype.

### Sample Collection.

Lumbar dorsal root ganglion (DRG) and spinal cord tissues were quickly dissected and snap-frozen in liquid nitrogen.

### RNA Extraction.

For DRG and spinal cord tissues, total RNA was extracted with phenol/chloroform and subsequently precipitated in isopropanol as described previously (71).

### Quantitative RT-PCR.

Reverse transcription was performed on 1 μg of RNA using high-capacity cDNA reverse transcription kit (Thermo Fisher), according the manufacturer's recommendation. Real time PCR was performed on a StepOne device using Taqman Gene Expression Master Mix (Thermo Fisher), following manufacturer's recommendations. Expression levels of the following genes were evaluated using comparative CT method (2-deltaCT): Tumor Necrosis Factor alpha (Tnfa, Assay ID Mm00443258_m1), Interleukin-6 (Il6, Assay ID Mm00446190_m1), Il1b (assay ID Mm00434228_m1); chemokine (C-C motif) ligand 2 (Ccl2, assay ID Mm00441242_m1). Transcript levels of PPIA (Mm02342430_m1) were used as endogenous control.

### Statistics

All data are presented as mean±SEM. Differences between groups were assessed using two-tailed Student's t-test, One-Way ANOVA followed by multiple comparison Tukey's post-hoc test or repeated measures two-way ANOVA using Prism (GraphPad software). Differences were considered statistically significant at p<0.05. The number of biologically independent experiments, sample size, p values, and statistical tests are all indicated in the main text or figure legends.

### Results

### A2AR Antagonism Limits Cisplatin-Induced Pain Hypersensitivity.

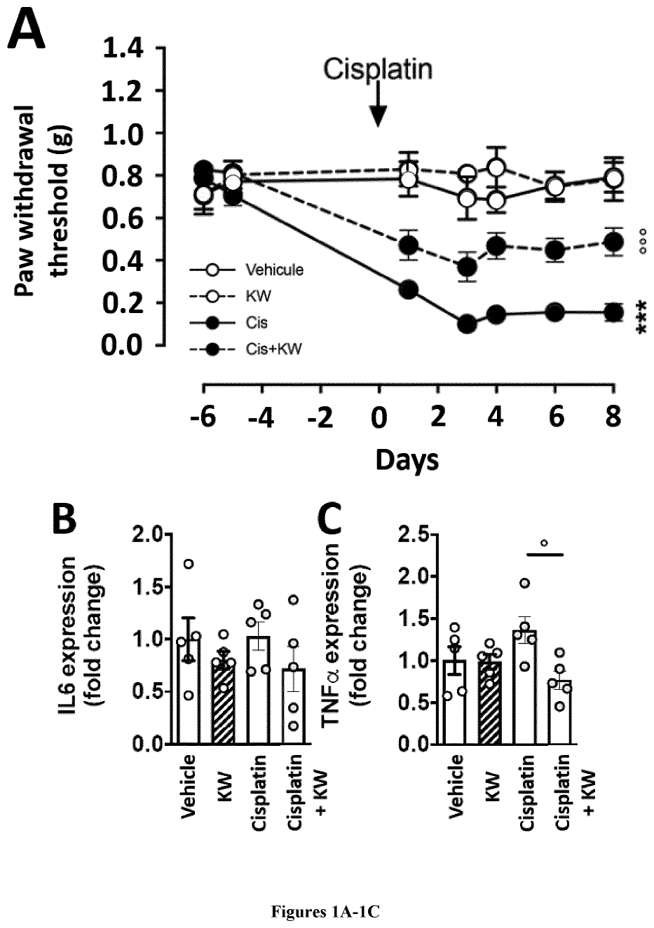

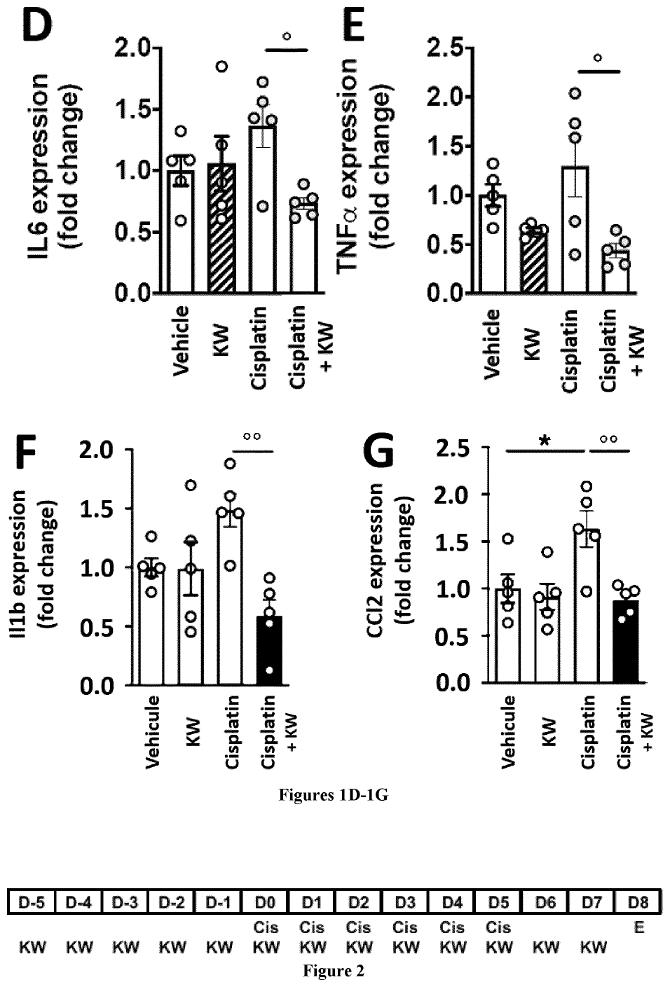

An important limitation in the therapeutic use of cisplatin is the development CIPN, in particular, pain hypersensitivity (Sisignano, 2014; Krukowski et al, 2017). To evaluate whether KW-6002 alleviates cisplatin-induced pain hypersensitivity and associated proinflammatory cytokines in the DRG (Dorsal Root Ganglion) and spinal cord, mice were treated with cisplatin and KW-6002 as described above (FIG. 2). Interestingly, treatment with KW-6002 significantly mitigated pain hypersensitivity (FIG. 1A). When we determined the effects of the A2AR antagonist on cisplatin-induced increase of Il6 and Tnf expression in DRG and spinal cord, we found that KW-6002 significantly downregulated Tnf in spinal cord as well as Il6 and Tnf expression in the DRG (FIG. 1B to FIG. 1E). We found that treatment with KW6002 significantly reversed the upregulation in the DRG of Il 1b and Ccl2 (FIGS. 1F and 1G), 2 cytokines known to contribute to CIPN (78, 79).

A2AR Antagonism Protects Against Cisplatin-Induced Nephrotoxicity A2A and CIPN, while Enhancing Tumor Growth Control in a Syngeneic Model of HPV+Squamous Carcinoma.

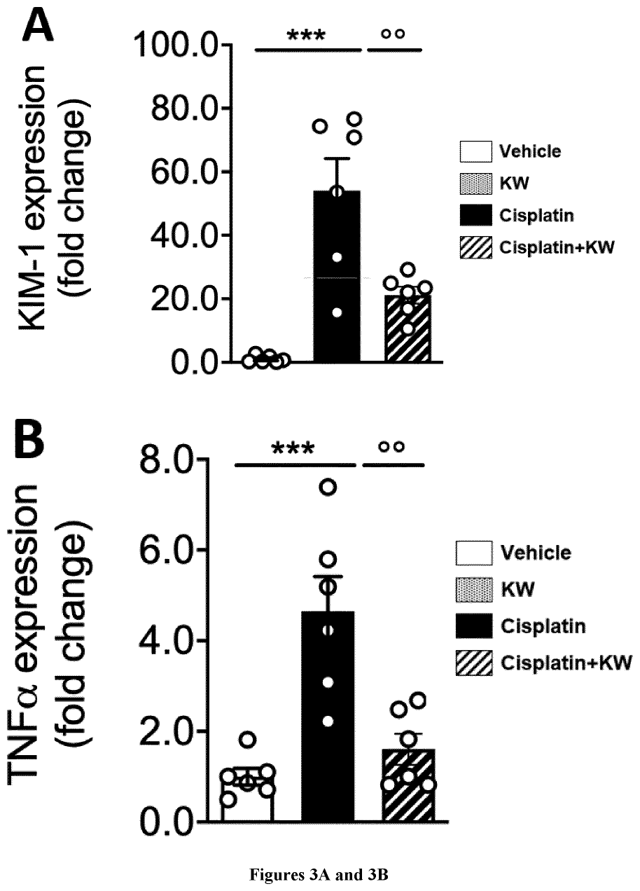

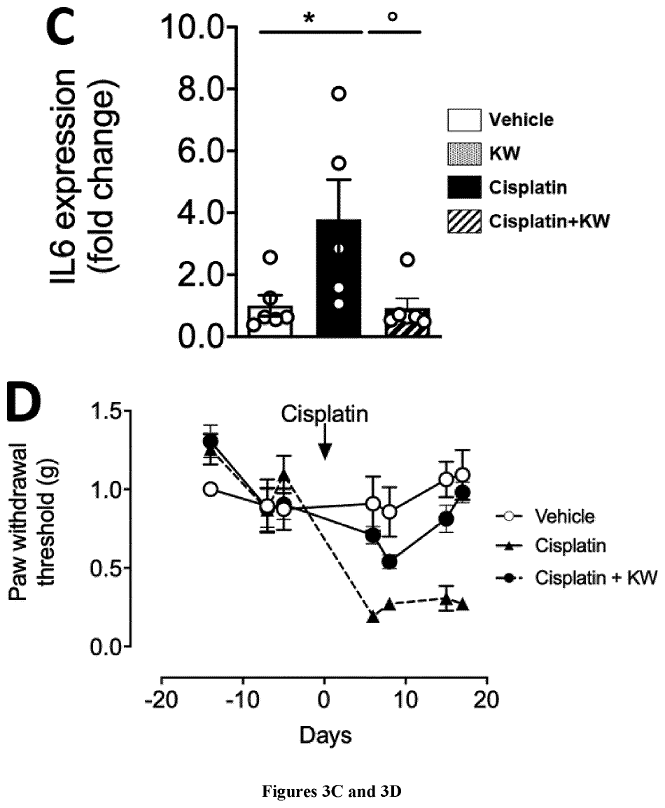

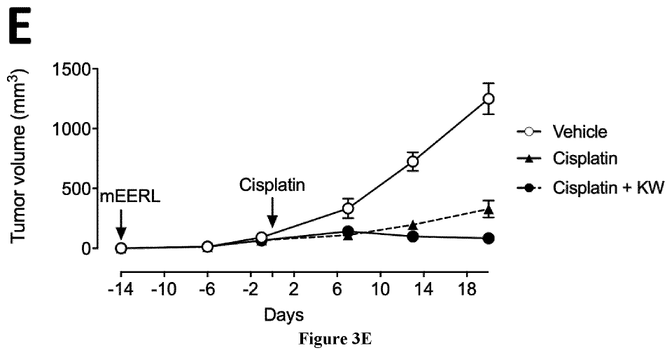

We validated the nephro- and neuro protective effects of KW6002 in a tumoral context using an additional cancer mouse model (80). Subcutaneous mEERL cells were injected into C57B16/J mice, which were then treated with cisplatin alone or in combination with KW6002. KW6002 administration in tumor-bearing mice limited indicators of renal toxicity (KIM-1, FIG. 3A) and expression of the inflammatory cytokines Tnf and Il6 (FIGS. 3B and 3C) induced by cisplatin. KW6002 also alleviated cisplatin-induced pain hypersensitivity in this model (FIG. 3D). Finally, KW6002 significantly potentiated tumor control by cisplatin (FIG. 3E). Overall, using this additional model with a different cancer etiology, the nephroprotective effect, the reduction of pain hypersensitivity, and the potentiation of tumor control were replicated, highlighting the promising therapeutic potential of A2AR inhibition.

These data supported that KW-6002 can also mitigate cisplatin-induced peripheral neuropathy.

### REFERENCES

Throughout this application, various references describe the state of the art to which this invention pertains. The disclosures of these references are hereby incorporated by reference into the present disclosure.
1. Dasari S, Tchounwou P B. Cisplatin in cancer therapy: molecular mechanisms of action. Eur J Pharmacol. 2014; 740:364-378.
2. Wang D, Lippard S J. Cellular processing of platinum anticancer drugs. Nat Rev Drug Discov. 2005; 4(4):307-320.
3. Miller R P, et al. Mechanisms of Cisplatin nephrotoxicity. Toxins (Basel). 2010; 2(11):2490-2518.
4. Rybak L P, Whitworth C A. Ototoxicity: therapeutic opportunities. Drug Discov Today. 2005; 10(19):1313-1321.
5. Staff N P, et al. Chemotherapy-induced peripheral neuropathy: A current review. Ann Neurol. 2017; 81(6):772-781.
6. Ciarimboli G. Membrane transporters as mediators of Cisplatin effects and side effects. Scientifica (Cairo). 2012; 2012:473829.
7. Manohar S, Leung N. Cisplatin nephrotoxicity: a review of the literature. J Nephrol. 2018; 31(1):15-25.
8. Oh G S, et al. Cisplatin-induced Kidney Dysfunction and Perspectives on Improving Treatment Strategies. Electrolyte Blood Press. 2014; 12(2):55-65.
9. Pabla N, Dong Z. Cisplatin nephrotoxicity: mechanisms and renoprotective strategies. Kidney Int. 2008; 73(9):994-1007.
10. Liu H E, et al. Multiple analytical approaches demonstrate a complex relationship of genetic and nongenetic factors with cisplatin- and carboplatin-induced nephrotoxicity in lung cancer patients. Biomed Res Int. 2014; 2014:937429.
11. Hamroun A, et al. Prevention of cisplatin-induced acute kidney injury: a systematic review and meta-analysis. Drugs. 2019; 79(14):1567-1582.
12. Cavaletti G, Marmiroli P. Chemotherapy-induced peripheral neurotoxicity. Nat Rev Neurol. 2010; 6(12):657-666.
13. Antonioli L, et al. Immunity, inflammation and cancer: a leading role for adenosine. Nat Rev Cancer. 2013; 13(12):842-857.
14. Chen J F, Eltzschig H K, Fredholm B B. Adenosine receptors as drug targets-what are the challenges?. Nat Rev Drug Discov. 2013; 12(4):265-286.
15. Haskó G, et al. Adenosine receptors: therapeutic aspects for inflammatory and immune diseases. Nat Rev Drug Discov. 2008; 7(9):759-770.
16. Jacobson K A, Gao Z G. Adenosine receptors as therapeutic targets. Nat Rev Drug Discov. 2006; 5(3):247-264.
17. Jacobson K A, Gao Z G, Matricon P, Eddy M T, Carlsson J. Adenosine A2A receptor antagonists: from caffeine to selective non-xanthines. Br J Pharmacol. 2020; 10.1111/bph.15103. doi:10.1111/bph.15103
18. Cunha R A. How does adenosine control neuronal dysfunction and neurodegeneration?. J Neurochem. 2016; 139(6):1019-1055.
19. Xiao C, Liu N, Jacobson K A, Gavrilova O, Reitman M L. Physiology and effects of nucleosides in mice lacking all four adenosine receptors. PLoS Biol. 2019; 17(3):e3000161.
20. Oyarzún C, Garrido W, Alarcón S, et al. Adenosine contribution to normal renal physiology and chronic kidney disease. Mol Aspects Med. 2017; 55:75-89.
21. Dwyer K M, Kishore B K, Robson S C. Conversion of extracellular ATP into adenosine: a master switch in renal health and disease. Nat Rev Nephrol. 2020; 16(9):509-524.
22. Lee H T, et al. A1 adenosine receptor activation inhibits inflammation, necrosis, and apoptosis after renal ischemia-reperfusion injury in mice. J Am Soc Nephrol. 2004; 15(1):102-111.
23. Grenz A, et al. The reno-vascular A2B adenosine receptor protects the kidney from ischemia. PLoS Med. 2008; 5(6):e137.
24. Park S W, et al. Proximal tubule sphingosine kinase-1 has a critical role in A1 adenosine receptor-mediated renal protection from ischemia. Kidney Int. 2012; 82(8):878-891.
25. Roberts V S, et al. The role of adenosine receptors A2A and A2B signaling in renal fibrosis. Kidney Int. 2014; 86(4):685-692.
26. Sheth S, et al. Adenosine receptors: expression, function and regulation. Int J Mol Sci. 2014; 15(2):2024-2052.
27. Gill A, et al. Protective effect of tonapofylline (BG9928), an adenosine A1 receptor antagonist, against cisplatin-induced acute kidney injury in rats. Am J Nephrol. 2009; 30(6):521-526.
28. Knight R J, Collis M G, Yates M S, Bowmer C J. Amelioration of cisplatin-induced acute renal failure with 8-cyclopentyl-1,3-dipropylxanthine. Br J Pharmacol. 1991; 104(4):1062-1068.
29. Nagashima K, Kusaka H, Karasawa A. Protective effects of KW-3902, an adenosine A1-receptor antagonist, against cisplatin-induced acute renal failure in rats. Jpn J Pharmacol. 1995 April; 67(4):349-57.
30. Day Y J, et al. Renal protection from ischemia mediated by A2A adenosine receptors on bone marrow-derived cells. J Clin Invest. 2003; 112(6):883-891.
31. Vincent I S, Okusa M D. Adenosine 2A receptors in acute kidney injury. Acta Physiol (Oxf). 2015; 214(3):303-310.
32. Garcia G E, et al. Adenosine A2A receptor activation prevents progressive kidney fibrosis in a model of immune-associated chronic inflammation. Kidney Int. 2011; 80(4):378-388.
33. Awad A S, et al. Adenosine A2A receptor activation attenuates inflammation and injury in diabetic nephropathy. Am J Physiol Renal Physiol. 2006; 290(4):F828-F837.
34. Garcia G E, et al. Adenosine A2A receptor activation and macrophage-mediated experimental glomerulonephritis. FASEB J. 2008; 22(2):445-454.
35. Holditch S J, et al. Recent advances in models, mechanisms, biomarkers, and interventions in cisplatin-induced acute kidney injury. Int J Mol Sci. 2019; 20(12):3011.
36. Oh C J, et al. Pyruvate dehydrogenase kinase 4 deficiency attenuates cisplatin-induced acute kidney injury. Kidney Int. 2017; 91(4):880-895.
37. Li S, et al. Reduced kidney lipoprotein lipase and renal tubule triglyceride accumulation in cisplatin-mediated acute kidney injury. Am J Physiol Renal Physiol. 2012; 303(3):F437-F448.
38. Sisignano M, et al. Mechanism-based treatment for chemotherapy-induced peripheral neuropathic pain. Nat Rev Neurol. 2014; 10(12):694-707.
39. Krukowski K, et al. HDAC6 inhibition effectively reverses chemotherapy-induced peripheral neuropathy. Pain. 2017; 158(6):1126-1137.
40. Santos N A, et al. Cisplatin-induced nephrotoxicity is associated with oxidative stress, redox state unbalance, impairment of energetic metabolism and apoptosis in rat kidney mitochondria. Arch Toxicol. 2007; 81(7):495-504.
41. Freitas-Lima L C, et al. PPAR-α deletion attenuates cisplatin nephrotoxicity by modulating renal organic transporters MATE-1 and OCT-2. Int J Mol Sci. 2020; 21(19):7416.
42. Cohen A, et al. Mechanism and reversal of drug-induced nephrotoxicity on a chip. Sci Transl Med. 2021; 13(582):eabd6299.
43. Aires I D, et al. Blockade of microglial adenosine A2A receptor suppresses elevated pressure-induced inflammation, oxidative stress, and cell death in retinal cells. Glia. 2019; 67(5):896-914.
44. Bortolatto C F, et al. Selective A2A receptor antagonist SCH 58261 modulates striatal oxidative stress and alleviates toxicity induced by 3-Nitropropionic acid in male Wistar rats. Metab Brain Dis. 2017; 32(6):1919-1927.
45. Leite M R, Wilhelm E A, Jesse C R, Brandão R, Nogueira CW. Protective effect of caffeine and a selective A2A receptor antagonist on impairment of memory and oxidative stress of aged rats. Exp Gerontol. 2011; 46(4):309-315.
46. Nobre H V Jr, et al. Caffeine and CSC, adenosine A2A antagonists, offer neuroprotection against 6-OHDA-induced neurotoxicity in rat mesencephalic cells. Neurochem Int. 2010; 56(1):51-58.
47. Thakur S, Du J, Hourani S, Ledent C, Li J M. Inactivation of adenosine A2A receptor attenuates basal and angiotensin II-induced ROS production by Nox2 in endothelial cells. J Biol Chem. 2010; 285(51):40104-40113.
48. Kelland L R. Preclinical perspectives on platinum resistance. Drugs. 2000; 59 Suppl 4:1-38.
49. Zsengellér Z K, Ellezian L, Brown D, et al. Cisplatin nephrotoxicity involves mitochondrial injury with impaired tubular mitochondrial enzyme activity. J Histochem Cytochem. 2012; 60(7):521-529.
50. Galgamuwa R, et al. Dichloroacetate prevents cisplatin-induced nephrotoxicity without compromising cisplatin anticancer properties. J Am Soc Nephrol. 2016; 27(11):3331-3344.
51. Cetin R, Devrim E, Kiliçoğlu B, Avci A, Candir O, Durak I. Cisplatin impairs antioxidant system and causes oxidation in rat kidney tissues: possible protective roles of natural antioxidant foods. J Appl Toxicol. 2006; 26(1):42-46.
52. Estrela G R, et al. MATE-1 modulation by kinin B1 receptor enhances cisplatin efflux from renal cells. Mol Cell Biochem. 2017; 428(1-2):101-108.
53. Nakamura T, et al. Disruption of multidrug and toxin extrusion MATE1 potentiates cisplatin-induced nephrotoxicity. Biochem Pharmacol. 2010; 80(11):1762-1767.
54. Kim D G, Bynoe M S. A2A adenosine receptor modulates drug efflux transporter P-glycoprotein at the blood-brain barrier. J Clin Invest. 2016; 126(5):1717-1733.
55. Menzies R I, Tam F W, Unwin R J, Bailey M A. Purinergic signaling in kidney disease. Kidney Int. 2017; 91(2):315-323.
56. Ohta A, et al. A2A adenosine receptor protects tumors from antitumor T cells. Proc Natl Acad Sci USA. 2006; 103(35):13132-13137.
57. Sitkovsky M V, et al. Hostile, hypoxia-A2-adenosinergic tumor biology as the next barrier to overcome for tumor immunologists. Cancer Immunol Res. 2014; 2(7):598-605.
58. Allard B, Allard D, Buisseret L, Stagg J. The adenosine pathway in immuno-oncology. Nat Rev Clin Oncol. 2020; 17(10):611-629.
59. Yu F, Zhu C, Xie Q, Wang Y. Adenosine A2A receptor antagonists for cancer immunotherapy. J Med Chem. 2020; 63(21):12196-12212.
60. Kawahara M, et al. Caffeine dose-dependently potentiates the antitumor effect of cisplatin on osteosarcomas. Anticancer Res. 2008; 28(3A):1681-1685.
61. Abe K, Yamamoto N, Hayashi K, Takeuchi A, Tsuchiya H. Caffeine citrate enhanced cisplatin antitumor effects in osteosarcoma and fibrosarcoma in vitro and in vivo. BMC Cancer. 2019; 19(1):689.
62. Chen J F, Cunha R A. The belated US FDA approval of the adenosine A2A receptor antagonist istradefylline for treatment of Parkinson's disease. Purinergic Signal. 2020; 16(2):167-174.
63. Sharp C N, et al. Repeated administration of low-dose cisplatin in mice induces fibrosis. Am J Physiol Renal Physiol. 2016; 310(6):F560-F568.
64. Sharp C N, Siskind L J. Developing better mouse models to study cisplatin-induced kidney injury. Am J Physiol Renal Physiol. 2017; 313(4):F835-F841.
65. Chaplan S R, et al. Quantitative assessment of tactile allodynia in the rat paw. J Neurosci Methods. 1994; 53(1):55-63.
66. Laumet G, et al. Cisplatin educates CD8+ T cells to prevent and resolve chemotherapy-induced peripheral neuropathy in mice. Pain. 2019; 160(6):1459-1468.
67. Pellegrini K L, et al. MicroRNA-155 deficient mice experience heightened kidney toxicity when dosed with cisplatin. Toxicol Sci. 2014; 141(2):484-492.
68. Aschauer L, et al. Expression of xenobiotic transporters in the human renal proximal tubule cell line RPTEC/TERT1. Toxicol In Vitro. 2015; 30(1 Pt A):95-105.
69. Vandenbussche C, et al. Tacrolimus-induced nephrotoxicity in mice is associated with microRNA deregulation. Arch Toxicol. 2018; 92(4):1539-1550.
70. Lemaire J, et al. Cadmium-induced renal cell toxicity is associated with microrna deregulation. Int J Toxicol. 2020; 39(2):103-114.
71. Vomelová I, et al. Methods of RNA purification. All ways (should) lead to Rome. Folia Biol (Praha). 2009; 55(6):243-251.
72. Dobin A, et al. STAR: ultrafast universal RNA-seq aligner. Bioinformatics. 2013; 29(1):15-21.
73. Anders S, et al. HTSeq—a Python framework to work with high-throughput sequencing data. Bioinformatics. 2015; 31(2):166-169.
74. Anders S, Huber W. Differential expression analysis for sequence count data. Genome Biol. 2010; 11(10):R106.
75. Love M I, Huber W, Anders S. Moderated estimation of fold change and dispersion for RNA-seq data with DESeq2. Genome Biol. 2014; 15(12):550.
76. Benjamini Y, Hochberg Y. Controlling the False Discovery Rate—a Practical and Powerful Approach to Multiple Testing. J R Stat Soc Ser B-Methodological. 1995; 57(1):289-300.
77. Zajączkowska R, et al. Mechanisms of Chemotherapy-Induced Peripheral Neuropathy. Int J Mol Sci. 2019 March; 20(6): 1451.
78. Illias A M, et al. Chemokine CCL2 and its receptor CCR2 in the dorsal root ganglion contribute to oxaliplatin-induced mechanical hypersensitivity. Pain. 2018; 159(7):1308-1316.
79. Kim H K, et al. Rolipram, a selective phosphodiesterase 4 inhibitor, ameliorates mechanical hyperalgesia in a rat model of chemotherapy-induced neuropathic pain through inhibition of inflammatory cytokines in the dorsal root ganglion. Front Pharmacol. 2017; 8:885.
80. Heussner M J, et al. A novel syngeneic immunocompetent mouse model of head and neck cancer pain independent of interleukin-1 signaling. Anesth Analg. 2021; 132(4):1156-1163.

1. A method for the treatment of neuropathy in a subject in need therefore comprising administering to the subject a therapeutically effective amount of a selective A2A Adenosine Receptor (A2AR) antagonist.

2. The method according to claim 1 wherein the neuropathy is a chemotherapy-induced peripheral neuropathy (CIPN).

3. The method according to claim 2 wherein the CIPN is induced by a chemotherapeutic drug.

4. The method according to claim 3 wherein the chemotherapeutic drug is cisplatin.

5. The method according to claim 1 wherein the selective A2A Adenosine Receptor antagonist is chosen among istradefylline (KW-6002) or a derivative thereof, MSX-3, SCH-58261, tozadenant (SYN 115), preladenant (SCH-420814), NIR178 (PBF-509), ciforadenant, AB928, AZD4635, EOS100850, Inupadenant (EOS-850), EXS21546, TT-10 and TT-53 and pharmaceutically acceptable salts thereof.

6. The method according to claim 1 wherein the selective A2A Adenosine Receptor antagonist is KW-6002.

7. The method according to claim 1 wherein the selective A2A Adenosine Receptor antagonist is administrated by oral or by parenteral administration.

8. A pharmaceutical composition comprising a therapeutically effective amount of a selective A2A Adenosine Receptor (A2AR) antagonist and a chemotherapeutic drug.

9. The pharmaceutical composition according to claim 8 wherein the selective A2A Adenosine Receptor (A2AR) antagonist is KW-6002 or a derivative thereof.

10. The pharmaceutical composition according to claim 8 wherein the chemotherapeutic drug is cisplatin.

11. The pharmaceutical composition according to claim 8, further comprising one or more pharmaceutically acceptable excipients.

12. (canceled)

13. (canceled)

14. The method of claim 5, wherein the selective A2A Adenosine Receptor antagonist is istradefylline (KW-6002) or a derivative thereof, MSX-3 or SCH-58261.
